(19) United States
(12) Patent Application Publication
(10) Pub. No.: US 2021/0157979 A1
(43) Pub. Date: May 27, 2021

(54) Systems and Methods for Extracting Form Information Using Enhanced Natural Language Processing

(71) Applicant: 3M INNOVATIVE PROPERTIES COMPANY, St. Paul, MN (US)

(72) Inventors: Amy A. Sheide, Salt Lake City, UT (US); Barbara C. Zellerino, Newburgh, IN (US)

(21) Appl. No.: 16/605,929

(22) PCT Filed: Apr. 18, 2018
(86) PCT No.: PCT/US2018/028061

§ 371 Date: Oct. 17, 2019

Related U.S. Application Data
(60) Provisional application No. 62/489,206, filed on Apr. 24, 2017.
(60) Provisional application No. 62/551,937, filed on Aug. 30, 2017.
(60) Provisional application No. 62/653,587, filed on Apr. 6, 2018.

(51) Int. Cl. G06F 40/205; G06F 40/289; G16H 10/60

(52) U.S. Cl. CPC G06F 40/205; G16H 10/60; G06F 40/289

(57) ABSTRACT

At least some aspects of the present disclosure direct to systems and methods of extracting medical entry information from medical documentation. A method comprises the steps of: identifying patient information needed for a predefined medical entry; finding the patient information in documents associated with the patient, wherein finding the patient information includes annotating the documents with a natural language processor to detect phrases and words corresponding to the patient information in the patient documents and analyzing the documents with a machine learning processor trained using the annotated documents to detect the patient information in the patient documents; and exporting the patient information found as medical entry fields.

### BACKGROUND

Many forms are filled every day. For example, healthcare visits and submissions often include many forms. Some or all of the form entries may be documented in one or more dispersed documents. As an example, when a healthcare provider interacts with a patient in a hospital setting, the provider typically memorializes the encounter, usually by typing or dictation. The provider may, for instance, memorialize the condition of the patient, the treatment plan, and what was done to the patient for treatment. Typically, the resultant encounter-related documentation is reviewed by documentation review specialists, who read through, update and request clarifications as needed to the encounter-related documentation. Once the patient is discharged the medical coders will apply the necessary coded information and the encounter can then be billed to the appropriate public or private payer.

Healthcare organizations also participate in the collection and submission of data for a variety of disease, procedures, and devices and collect data elements in a registry. Process registries are used to understand patient populations and to drive protocols and best practices with the goal of promoting evidence based clinical care. This process is primarily manual and requires human review of medical documentation, such as the encounter-related documentation described above, to abstract the required data elements from that documentation. The data elements, herein referred to as form entry fields, are often defined by outside organizations, such as governing bodies. Despite the definition received from the organizations, the complexity and variability of the data within clinical documentation can make the data needed for each registry entry difficult to find, interpret and produce in an efficient, reliable and scalable manner.

### SUMMARY

At least some aspects of the present disclosure direct to a method of extracting form entries, the method comprising: receiving one or more documents; identifying fields, by a processor, needed for a predefined form entry; and generating a plurality of field records based on the documents, wherein each of the plurality of field records corresponds to one of the fields and includes a field value and one or more evidences, wherein for each of the plurality of field records, extracting the one or more evidences from the documents with a natural language processor to detect phrases and words corresponding to the field in the documents; analyzing the one or more evidences with a machine learning processor; and suggesting the field value based on the one or more evidences, wherein at least one of the one or more evidences is a negating evidence.

At least some aspects of the present disclosure direct to a method of extracting medical entry information from medical documentation. The method comprises the steps of: identifying patient information needed for a predefined medical entry; finding the patient information in documents associated with the patient, wherein finding the patient information includes annotating the documents with a natural language processor to detect phrases and words corresponding to the patient information in the patient documents and analyzing the documents with a machine learning processor trained using the annotated documents to detect the patient information in the patient documents; exporting the patient information found as medical entry fields.

At least some aspects of the present disclosure directs to a method of extracting medical entry information from medical documentation, The method comprises the steps of: identifying patient information needed for a predefined medical entry; finding the patient information in documents associated with the patient, wherein finding the patient information includes analyzing the documents with a machine learning processor trained using annotated documents to detect the patient information in the patient documents; displaying the patient information; receiving input selecting the patient information to export; and exporting the selected patient information as medical entry fields.

At least some aspects of the present disclosure direct to a method of training a machine learning processor. The method comprises the steps of: identifying, in a predefined set of documents annotated with codes, all the codes that pertain to diagnoses or procedures of interest; identifying all instances in the predefined set of documents of the identified codes; creating a machine learning model to identify characteristics of each document that led to annotation of the document with the identified codes; training the machine learning processor based on the machine learning model; and testing the machine learning processor on the predefined set of documents.

At least some aspects of the present disclosure direct to a method of training a machine learning processor. The method comprises the steps of: identifying fields in a medical entry; applying natural language processing to annotate information relevant to the medical entry fields in a pre-defined set of documents; applying rule-based processing to annotate information used to derive information relevant to the medical entry fields in a pre-defined set of documents; and training the machine learning processor as a function of the annotated information.

At least some aspects of the present disclosure direct to a method of training a machine learning processor. The method comprises the steps of: identifying fields in a medical entry; applying natural language processing to annotate information relevant to the medical entry fields in a pre-defined set of documents; applying rule-based processing to annotate information used to derive information relevant to the medical entry fields in a pre-defined set of documents; pretraining the machine learning processor as a function of the annotated information; and verifying the pretrained machine learning processor against the pre-defined set of documents.

### BRIEF DESCRIPTION OF DRAWINGS

FIG. 1A illustrates an example document analysis and data extraction system.

FIG. 1B illustrates an alternate example document analysis and data extraction system.

FIG. 2 illustrates an example flow diagram for document analysis and data extraction.

FIG. 3 illustrates representative QT measurement language samples.

FIG. 4 illustrates an example user interface used to surface data and context from medical documentation by searching by patient, document, encounter or other text.

FIG. 5A illustrates an example method of analyzing documents to extract information for a form.

FIG. 5B illustrates an example method of analyzing patient medical documentation to extract information relevant to medical data collection forms.

FIG. 6A illustrates an example display of registry relevant information discovered in a search of patient medical documentation, and the context within which the information was found.

FIG. 6B illustrates an alternate example display of registry relevant information discovered in a search of patient medical documentation, and the context within which the information was found.

FIG. 7A illustrates an example method of training a machine learning processing system to analyze and extract information from documents.

FIG. 7B illustrates an example method of training a machine learning processing system to analyze and extract information from medical documentation.

FIG. 8 illustrates an example method of analyzing patient medical documentation based on natural language processing, rule-based processing and machine learning to extract information relevant to registry data collection forms.

FIGS. 9A and 9B illustrate an organization of clinical and diagnostic codes in one example of a Healthcare Data Dictionary (HDD).

FIG. 10 illustrates an example user interface used select documents used to train a machine learning processor.

FIGS. 11-13 illustrate example user interfaces used to analyze and annotate documents used to train the machine learning processor.

FIG. 14A illustrates an example flow diagram to extract evidences/contexts for a form field entry.

FIGS. 14B-E illustrate user interfaces for form field entry and evidence/context information.

FIG. 15A illustrates an example data structure that can be used to represent form entries.

FIG. 15B illustrates an example user interface for managing forms.

FIG. 16 illustrates an example user interface allowing a user to enter a search term to do a text search.

### DETAILED DESCRIPTION

Numerous forms are filled out manually every day. In many cases, the form entries are documented in one or more disperse documents. At least some aspects of the present disclosure direct to the methods and systems of extracting form entries of a predefined forms from one or more documents. As one example, such an information exaction system can be used in the medical field. A patient's encounter with a healthcare organization is usually initially documented by an admitting physician, attending physician, or by an emergency department physician in the emergency department, who may dictate the patient's condition, treatments, etc. In addition, there are other medical departments and software systems that contribute to the documentation for a healthcare encounter. The encounter related documentation may be used to update an electronic health record (EHR) associated with the patient. The electronic health record is also known as an electronic medical record (EMR).

Most hospitals have an EHR system, containing inpatient and outpatient encounter information for patients. The EHR includes the information about each patient, in digital format. EHRs contain the medical record for the patient; the information contained in the EHR for each patient is usually, however, spread across multiple documents and reports, and may lack a cohesive, validated and updated summary of the patient and his or her conditions. A physician spends a significant amount of time reviewing EHRs and determining treatment plans, issuing orders and documenting on their patients.

Encounter-related documentation may be used in other ways as well. For instance, the encounter-related documentation may be reviewed by billing specialists or medical coders to determine the most effective combination of billing codes for each encounter. The coding process is usually either done automatically using natural language processing (NLP) algorithms, or by professional coders reviewing the encounter related documentation (or via some version in-between). Between EHRs, billing reports and other documentation, health care providers accumulate a plethora of patient-related documentation. That documentation can be mined for medical record information associated with that patient. In some cases, the medical record information is to be used to fill out a medical entry having predefined entries, such as registries.

The systems and methods disclosed herein show examples of systems designed to facilitate efficient extraction of information from documentation and to simplify the transfer of such information to data collection systems. In some embodiments, such systems and methods are used to process medical information and for medical data collection systems. This may result in more accurate and timely submissions to forms including a number of form entries. In some cases, the form entries are medical entries. In some examples, the form entries are registries. In some cases, such systems and methods use enhanced NLP.

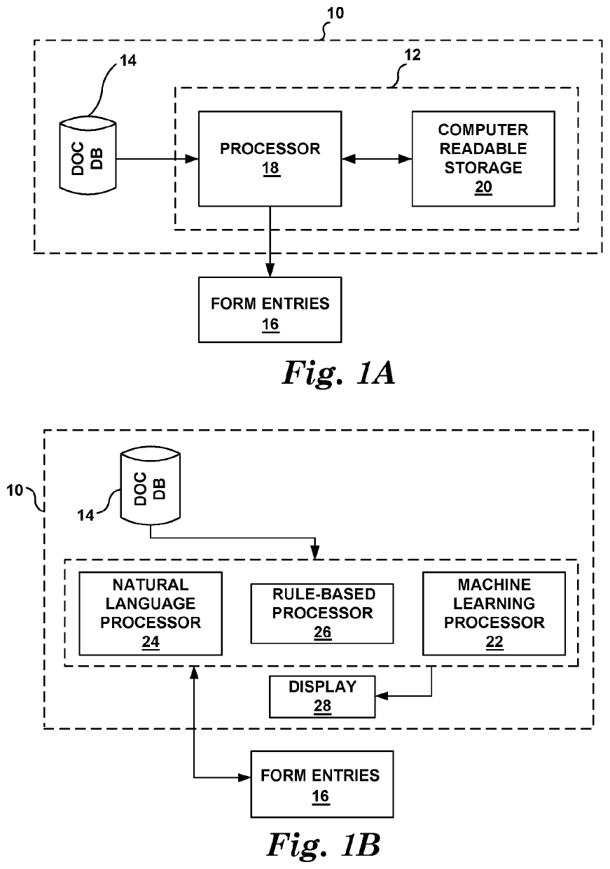

FIG. 1A illustrates an example document analysis and data extraction system. Document analysis and data extraction system 10 includes a computing system 12 connected to a document database 14. Computing system 12 analyzes documents in document database 14 to extract data relevant to one or more form entries 16. In some cases, the form entries 16 are medical entries. In some cases, the form entries 16 are medical registries. In many cases, the form entries are predefined. In one example approach, the documents stored in document database 14 include electronic health records, encounter-related documentation and documents such as problem lists and billing records that are derived from encounter-related documentation. In some cases, the documents are associated with a subject across a time period. As one example, the documents are associated with a law suit. In some cases, the documents are associated with the patient across the continuum of care.

In one example approach, computing system 12 includes one or more processors 18 connected to computer readable storage 20. In one such example approach, instructions stored in computer readable storage 20, when executed by the one or more processors 18, execute one or more of natural language processing of the documents to identify medical entry relevant information, rules processing of document content to derive form entry relevant information and machine learning processing of document content to identify form entry relevant information.

FIG. 1B illustrates an alternate example document analysis and data extraction system. Document analysis and data extraction system 10 includes a document database 14 connected to a machine learning processor 22 and a natural language processor 24. Both machine learning processor 22 and natural language processor 24 analyze documents in document database 14 to extract data relevant to one or more form entries 16. In one example approach, the documents stored in document database 14 include electronic health records, encounter-related documentation and documents such as problem lists and billing records that are derived from encounter-related documentation. In the example approach of FIG. 1B, machine learning processor 22 transmits form entry relevant data found in each document to display 36, along with the context (e.g., as evidences) in which it found the information. Similarly, natural language processor 24 transmits form entry relevant data found in each document to display 36, along with the context in which it found the information.

In some embodiments, machine learning processor 22 provides an opportunity to determine patterns of diagnoses that may not be readily apparent. For instance, a combination of test results and diagnostic codes examined across a large body of medical documents covering a broad patient population may reveal trends where healthcare professionals arrive at diagnoses despite test results that indicate a typical diagnostic threshold has not been met. Similarly, the review of the documents by machine learning processor 22 might suggest that healthcare professionals are making, or should be making, a diagnosis at a lower threshold in the presence of certain combinations of diagnostics codes.

In one example approach, natural language processor 24 analyzes each document looking for variations of key words and phrases. For example, natural language processor 24 analyzes each document looking for information specific to, for instance, one or more SNOMED codes, one or more ICD codes. In one example, if a given term is found, that term may be suggestive of a corresponding SNOMED code. The information is therefore associated with the term. In one embodiment, this association is facilitated by creating a new annotated version of the document in a markup language that allows for the imbedding of metadata with terms, such as HTML, or some variant of XML. Natural language processing in general and its application to the computer-assisted coding of medical record data are described by Wolniewicz in Computer-assisted Coding and Natural Language Processing, https://multimedia.3m.com/mws/media/756879O/3m-cac-and-nlp-white-paper.pdf. the description of which is incorporated herein by reference. Wolniewicz discusses the use of tokenization, sentence and structure detection, part-of-speech (POS) tagging, normalization, named entity resolution, parsing, negation and ambiguity detection and semantics in natural language processing of medical documents. Wolniewicz also describes the use of the Unstructured Information Management Architecture (UIMA) as an appropriate technical platform used to supply these capabilities.

In one example approach, machine learning processor 22 implements statistical natural language processing. Statistical NLP means processor 22 learns the mappings for the NLP components as statistical relationships by processing many examples. The accuracy of a statistical model goes up with the volume of data available for learning. In fact, the performance of a deployed system 10 will improve after deployment as the system learns the codes most often selected. Statistical methods, however, required a very large annotated data set to use for training. In one such example approach, machine learning processor 22 is implemented on the UIMA software platform, a standardized and integrated NLP solution.

In one example approach, machine learning processor 22 implements an algorithm that examines “skip-grams” of tokens from medical documents and builds a “trie” data structure (also referred to as a prefix tree) via the skip-grams. Machine learning processor 22 may determine, based on the nodes of the trie, rules for associating form entry information with medical documents. Negative sampling models and models that treat documents as bags of words may be used as well.

In one example approach, machine learning processor 22 parses documents into tokens and then analyzes the tokens to generate skip-grams. A skip-gram is a particular way of modeling language. A skip-gram is based on a construct referred to as an n-gram. An n-gram is a consecutive subsequence of length n of some sequence of tokens w□ . . . wₙ. A k-skip-n-gram is a length-n subsequence having components that occur at distance at most k from each other. As an example, for the phrase “the quick brown fox jumps over the lazy dog,” the set of all 1-skip-2 grams comprises: “the brown,” “quick fox,” “brown jumps,” “fox over,” “jumps the,” “over lazy,” and the dog,” as well as all the 2-grams (also referred to as bigrams), e.g., “the quick,” “quick brown,” etc. Skip-grams may be more useful relative to n-grams for analyzing word data due to the data sparsity associated with n-grams.

Machine learning processor 22 then builds a trie data structure by adding nodes having skip-grams one layer at a time. In one such approach, the trie data structure includes a set of nodes in which each node of the tree represents a string. The path from a leaf node to the root of the tree represents the co-occurrence of a set of strings. In one example approach, the trie has a null root node (i.e. a node having a null string as its value); each node is associated with a skip-gram and each additional level of depth within the trie corresponds to an increase by one in the number of the skip-grams at that level of the trie (relative to the skip-grams at the previous (parent) depth level of the trie). So, the first level of the trie includes nodes comprising skip-grams of size 1 (unigrams), the second level of the trie includes nodes comprising skip-grams of size 2 (bigrams), and so on.

Machine learning processor 22 then analyzes and prunes the nodes. During the pruning process, machine learning processor 22 examines and removes nodes from the trie to reduce the search space and memory consumption associated with the nodes. After pruning, machine learning processor 22 examines nodes from a current level of the tree that were not pruned for possible output as rules that associate a form entry field with a skip-gram having a set of tokens. In another example approach, machine learning processor 22, after pruning, examines nodes that were not pruned from a current level of the tree for possible output as rules that associate a medical entry field (or a condition or procedure code) with a skip-gram having a set of tokens.

After populating a level of the trie with nodes, machine learning processor 22 then examines the remaining nodes for potential output as rules. As an example, machine learning processor 22 may output a node as a rule if a probability of that rule exceeds a specified output threshold probability. The outputted rule may consist of the skip-gram set of features (e.g., a feature set for the skip-gram) that map to a specified billing code. The set of features or feature set of a skip-gram may include one or more combinations of tokens that may be available from the skip-gram.

Once machine learning processor 22 outputs any rules, machine learning processor 22 generates one or more bloom filters corresponding to the nodes of the trie. The bloom filter is similar to a hashing function, and is a memory-efficient way that a computing device can use to determine whether an element is a member of a set of elements. A bloom filter cannot definitively indicate whether an item is a member of a set. However, a bloom filter can definitively indicate whether an item is not a member of a set.

After generating bloom filters for the current depth level of the trie, machine learning processor 22 begins populating the next level of the trie, and determines, using the bloom filters generated for the previous level of the trie, whether a candidate skip-gram node for addition to the trie is a potential member of any of the existing skip-gram sets of the trie. If the candidate node, to be added, is potentially a member of at least one of the existing sets of skip-grams, machine learning processor 22 adds the node comprising the candidate skip-gram to the next level of the trie. If machine learning processor 22 determines that the candidate node is not a member of any skip-gram nodes of the previous depth level, machine learning processor 22 prunes the candidate skip-gram node, and does not add the node to the trie. Machine learning processor 22 continues iteratively pruning skip-gram nodes, outputting rules, and adding layers to the trie until all skip-grams having the maximum skip-gram window size have been analyzed and either added or pruned.

In some examples, if applying a medical code using an outputted medical coding rule has a probability that exceeds a certain probability threshold, a computing system consistent with this disclosure may automatically apply the rule to a medical document, i.e. may automatically apply the medical code associated with the rule to the medical document. In some examples, if an outputted medical coding rule does not have a probability that exceeds the threshold, there may be a risk that automatically associating a medical code with a medical document may be erroneous. Thus, in the cases where the probability does not exceed the threshold, machine learning processor 22 may indicate and/or a medical coder may still manually review medical documents to which coding rules and their associated medical codes have been automatically applied.

In some embodiments, natural language processor 24 is also connected to rule-based processor 26. Rule-based processor 26 receives from natural language processor 24 indications of data found in each document that may be used to derive data relevant to one or more form entries 16, along with the context in which the data was found. Rule-based processor 26 derives data relevant to one or more form entries 16 from the information received from natural language processor 24 and sends the derived data, along with the context in which the data used was derived, to display 36. In one example approach, display 36 displays the data and context received from each of machine learning processor 22, natural language processor 24 and rule-based processor 26 such that an aggregator can select the information to export to form entry 16.

For example, an analyst may determine that a registry, as an example of a form, expects to be notified of all instances of long QT syndrome in its patient population. Long QT syndrome may be an indication that a pacemaker is needed. The analyst decomposes the definition of long QT syndrome to identify all instances of long QT syndrome. This involves identifying all the variations of the phrase and can involve mapping of variations, word order disambiguation, acronym disambiguation and noncontiguous phrase parsing. In addition, the analyst identifies information that can be used to derive an indication of long QT syndrome. For instance, a finding that the patient's QT interval is greater than 460 msec is generally accepted as an indication of long QT syndrome. The analyst develops a rule to be applied by rule-based processor 26 that looks for QT intervals greater than 460 msec in a document and generates a long QT syndrome indication for that document.

In one example approach, each condition is mapped to one or more diagnostic codes. For instance, long QT syndrome may be mapped to a given diagnostic code. In one such example approach, rule-based processor 26 maps all relevant diagnostic codes to fields in each medical entry. Rule-based processor 26 therefore includes one or more rules mapping diagnostic codes associated with long QT syndrome to long QT syndrome. Rule-based processor 26 would, therefore, include a rule equating a QT interval greater than 460 msec as an indicator of long QT syndrome and a rule determining that one or more diagnostic codes are indicators of long QT syndrome. In one example approach, rule-based processor 26 also includes one or more rules mapping billing codes associated with long QT syndrome to long QT syndrome.

In one example approach, natural language processor 24 applies the decomposed definition of each piece of data relevant to the medical entry to identify such data and to identify information that can be used to derive data relevant to the medical entry. Rule-based processor 26 receives from natural language processor 24 information that can be used to derive data relevant to the registry, along with the context in which the data was found. Rule-based processor 26 then applies one or more rules to derive the data relevant to medical entry 16 from the information received from natural language processor 24.

In some cases, data the medical entry 16 are fed into the document analysis and data extraction system 10 for further machine learning processing, for example, to improve data filters, improve data analytics, to refine rules used by the rule-based processor 26, and the like.

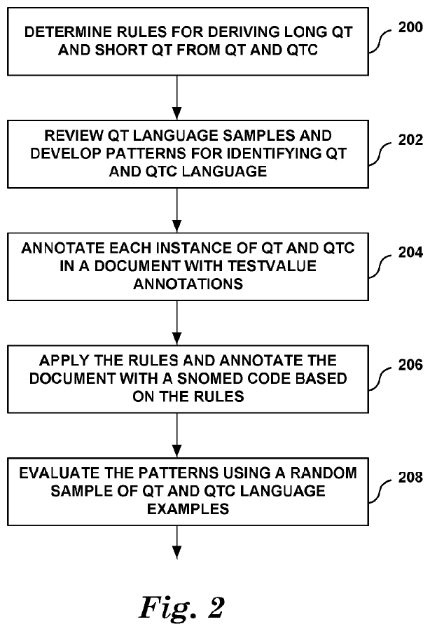

FIG. 2 illustrates an example flow diagram for medical document analysis and data extraction, including rules for determining long QT syndrome (SNOMED 9651007) and short QT syndrome (SNOMED 698272007) from corrected and uncorrected QT interval measurements. An analyst determines rules for deriving Long QT and short QT from QT and QTc. (100) In one example approach, the rules for determining long QT syndrome from corrected and uncorrected QT interval measurements are:

Long QT (SNOMED 9651007) should be coded when any of the following are true:
Corrected QT (QTc)>440 ms for adult men
Corrected QT (QTc)>460 ms for adult women
Non-corrected QT>500 ms for either gender
   Short QT (SNOMED 698272007) should be coded when:
QT is <=300 ms regardless of gender.
   In this example approach, there is no equivalent definition for corrected QT for short QT.

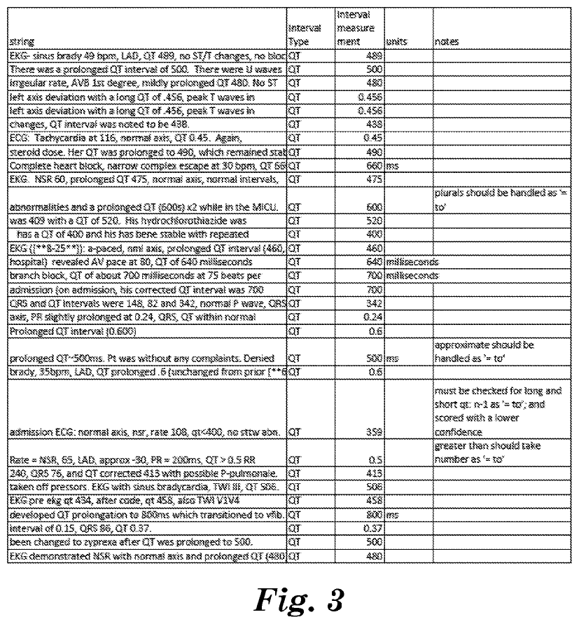

QT measurement language examples (such as shown in FIG. 3) are reviewed and patterns developed for detecting an acceptable percentage of QT measurement values. (202) In one example approach, the patterns are defined to capture 100% of QT and QTc values in a corpus of documents in which observations are always formatted the same way.) In one example approach, a new UIMA annotation type (TestValue(testName, Measurement)) is defined and used to annotate instances of QT and for QTc in documents. This UIMA type identifies the numeric result of a test, such as QT/QTc interval, BMI, blood pressure, etc. As noted above, it contains the following fields:

testName—The normalized name of the test, such as “qt” or “qtc”. These should be stored in a constants file and on a Wiki page.

measurement—A reference to a Measurement annotation containing the test value.

The annotation covers the test name only, not the measurement value. In the example “QT of 400 ms”, the TestValue will cover “QT” and the Measurement will cover “400 ms”. In the example “QT of 300”, the Measurement will cover “300”. The units are implied in this case.

System 10 receives the patterns defined for identifying QT and QTc, identifies instances of QT and QTc in documents based on the patterns and annotates each instance with a TestValue annotation. (204) In one example approach, system 10 iterates through all TestValue annotations with a testName of “qt” or “qtc”. All regions of a document are considered. Rule-based processor 26 applies the rules defined above to the values in the TestValue annotations and generates a SNOMED code when one or the rules is met. The document is then annotated with the SNOMED code. (206) In some example approaches, care is taken to ensure that the SNOMED code's evidence covers both the TestValue and the Measurement.

In one example approach, the patient's gender is read from the metadata associated with the document.

System 10 then evaluates the approach using a random sample of QT language examples. (208) In one example approach, a human identifies the documents that include a numeric QT or QTc value, and the documents where the QT and QTc values indicate long or short QT. This set of results is compared to the output from system 10. In one example approach, the evaluation passes if:

(a) System 10 identifies a QT or QTc value within an acceptable percent of the human-identified examples; and

(b) Whenever system 10 identifies a QT or QTc value, it identifies the following with 100% accuracy:
The correct QT or QTc value
Whether it is a QT vs. QTc
The “Long QT” or “Short QT” SNOMED code, if applicable

In one example approach, the test is repeated on a corpus of documents in which observations are always formatted the same way. System 10 is expected to detect 100% of the QT and QTc values identifies by a human analyzing the same data. In one example approach, the difference in thresholds for men vs. women is checked using a corpus of documents that includes patient gender.

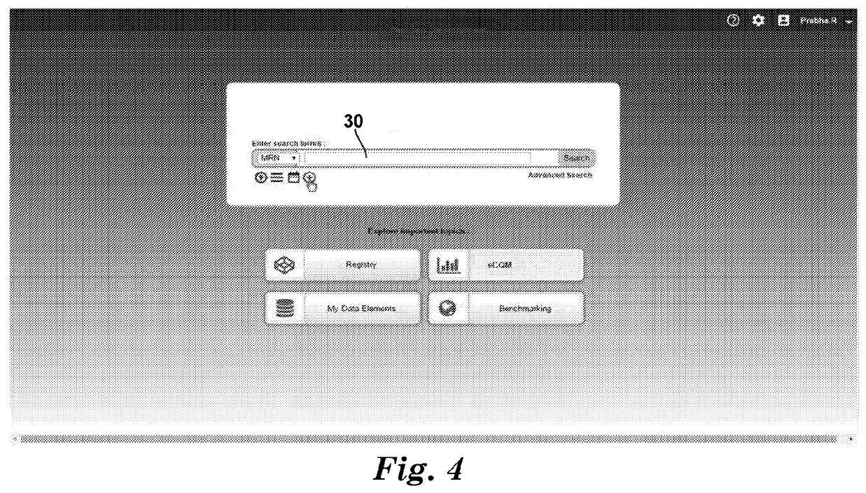

FIG. 4 illustrates an example user interface used to surface data and context from medical documentation by searching by patient, document, encounter or other text. A person preparing a registry entry would enter the patient's name in search field 30 and institute a search for all medical documentation for the patient in document database 14.

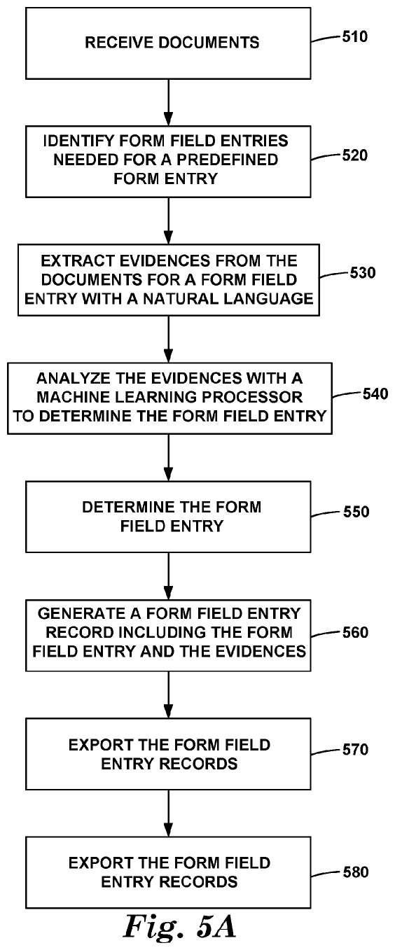

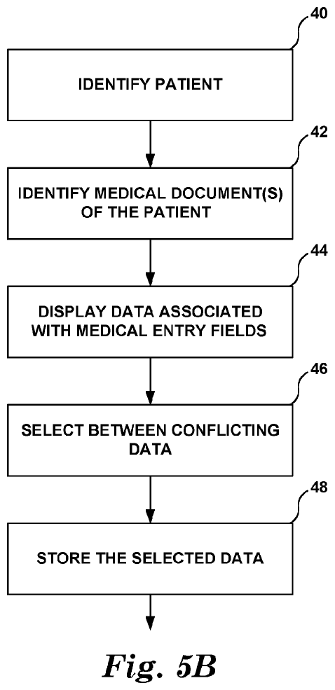

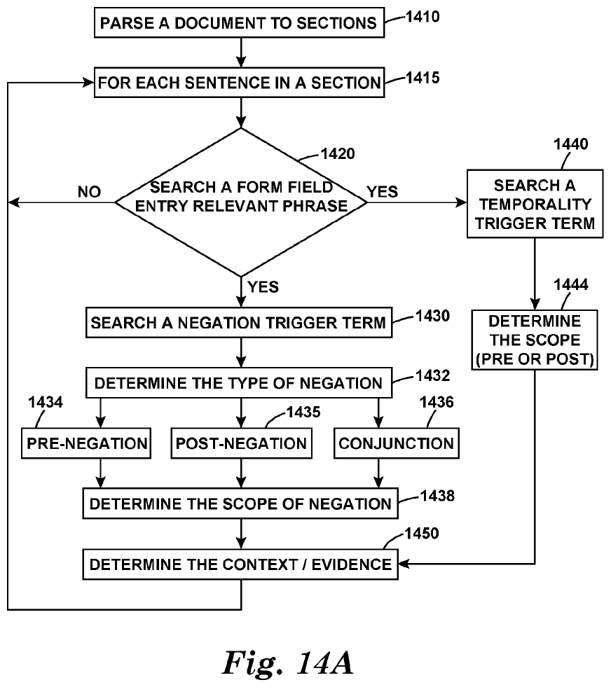

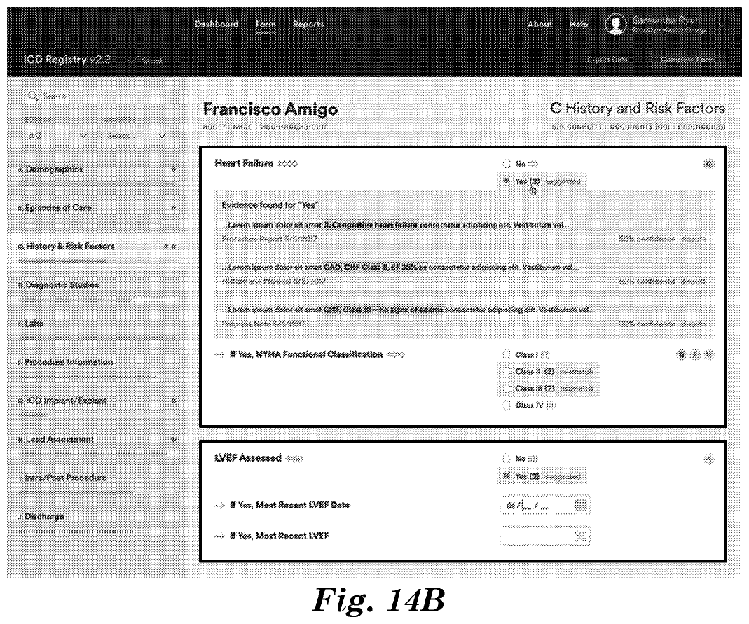

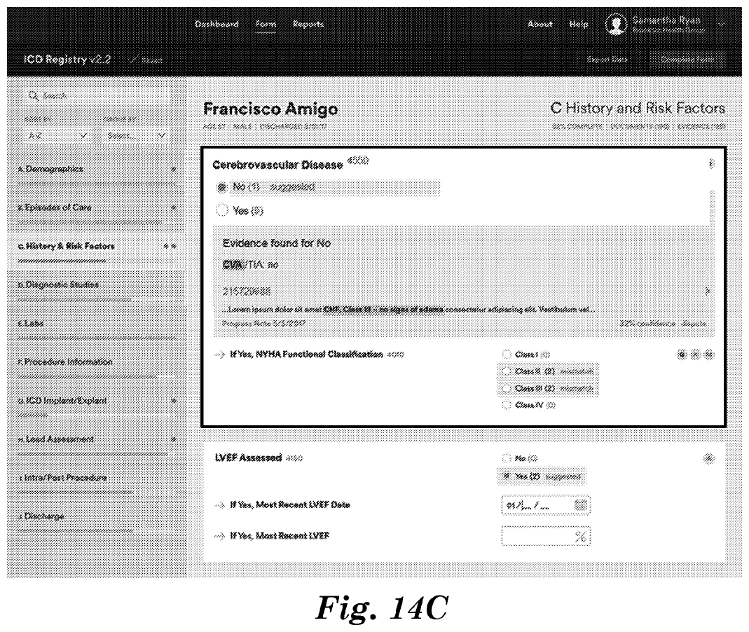

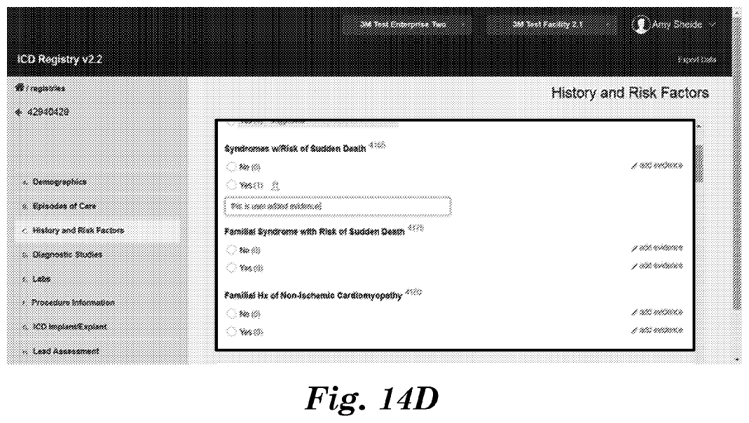

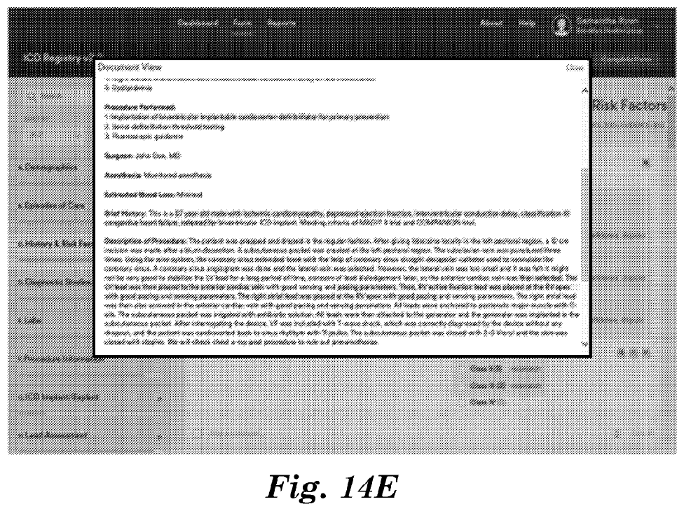

FIG. 5A illustrate an example flow diagram of analyzing documents to extract information for a form implemented on a computer system. Some of the steps are optional in this flow diagram. The computer system receives documents of a subject, for example, a patient, a case, and the like. (510) The computer system identifies form entries needed for a predefined form. (520) The computer system first extract evidences from the documents for a form field entry with a natural language processor. (530) The system then analyzes the evidences with a machine learning processor to determine the form field entry. (540) The computer system determines or suggests the form field entry based on the analysis. (550) For example, the form field entry is generated based on the most recent evidence. As another example, the form field entry is generated based on the supporting evidence(s). As yet another example, the form field entry is generated based on the negating evidence(s). For each identified form field entry, the computer system will create a form field entry record, which includes the content for the form field entry and the evidences for the form field entry. (560) After the form field entry records are generated, the computer system may export the field records. (570) In some cases, the computer system may display the form field entry records on a graphical user interface, for example, as illustrated in FIG. 14B. (580) The evidence/context extraction methods are describe in more details below.

FIG. 5B illustrates a method of analyzing patient medical documentation to extract information relevant to medical data collection forms. A person preparing a medical entry identifies the patient (by, for example, entering the patient's name in search field 30). (40) System 10 identifies medical documents associated with the patient and determines, for each document, if there is medical entry relevant information in the document. (42) The medical entry relevant information identified across all the documents is displayed (44) and the person preparing the medical entry selects the data to be used for the medical entry. (46) The data selected is then exported to be used to complete the medical entry for the patient. (48)

In one example approach, the medical entry relevant information is associated with one or more diagnostic or clinical codes and the data selected is mapped as a function of the diagnostic codes to medical entry field entries before being exported. Such an approach simplifies adding or changing codes associated with clinical conditions. In one example approach, proprietary clinical and billing codes based on, for example, 3M™ Healthcare Data Dictionary (HDD) content. Such an approach may provide more granularity and flexibility in identifying clinical conditions than approaches such as a coding based on, for example, SNOMED CT.

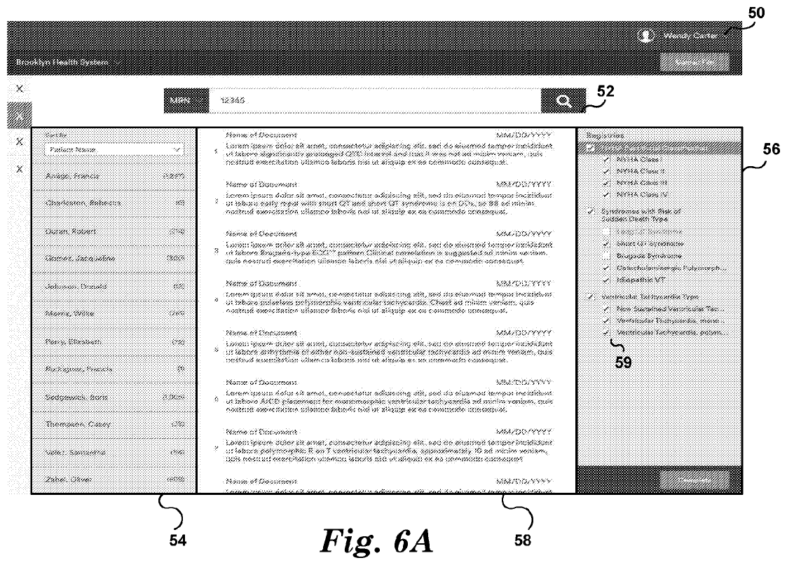

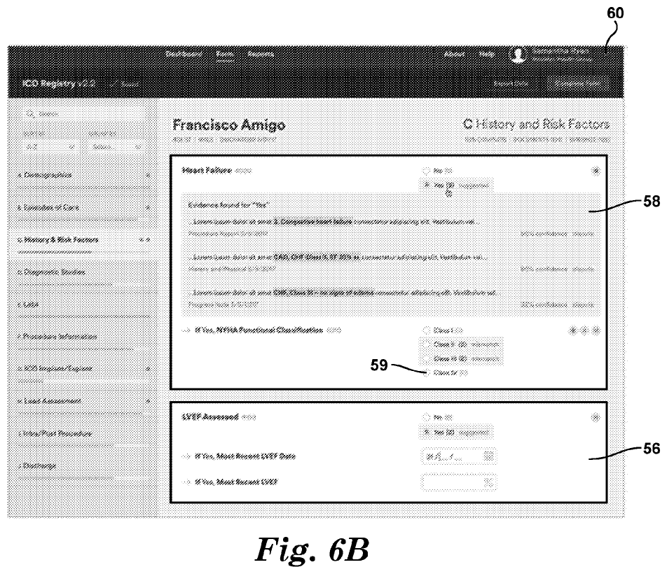

FIG. 6A illustrates an example display of medical entry (e.g., registry) relevant information discovered in a search of patient medical documents, and the context within which the information was found. As illustrated, FIG. 6A includes a registry data selection display 50 that has a search box 52, a sort by selector box 54, a registry information box 56 and a context display box 58. Registry relevant information is shown clustered by type in registry information box 56. Check off boxes 59 in registry information box 56 allow the person preparing a registry entry to select the information to be included in the registry entry. As can be seen in FIG. 6A, a review of all medical documents for a patient will often find conflicting information. In the example of FIG. 6A, the patient has, at different times, been coded as NYHA Class I, NYHA Class II, NYHA Class III and NYHA Class IV. The person preparing the registry entry may have to review the context information in context display box 58 to determine the current classification. In some example approaches, by clicking on the context entry in context display box 58, the person preparing the registry entry is taken to the underlying document for further review.

In one example approach, the person preparing the registry entry selects data to be included in the registry entry and indicates “Complete” when finished. In one such example approach, a check is made on receiving a “Complete” to determine if there are any conflicting entries and, if so, an error message is displayed. The person preparing the registry entry may simply address the errors and indicate “Complete” when finished.

FIG. 6B illustrates an alternate example display of registry relevant information discovered in a search of patient medical documents, and the context within which the information was found. FIG. 6B includes a registry data selection display 60 that has a registry information box 56 and a context display box 58. Registry relevant information is shown clustered by type in registry information box 56. Check off boxes 59 in registry information box 56 allow the person preparing a registry entry to select the information to be included in the registry entry. As can be seen in FIG. 6B, all conflicting information has been resolved and the appropriate check boxes 59 selected. In contrast to the example of FIG. 6A, the context information displayed in context display box 58 includes a confidence level indicating the confidence level calculated for the registry relevant information associated with the context information. The confidence level information may be used by the person preparing the registry entry, for instance, to select between conflicting data. In some example approaches, by clicking on the context entry in context display box 58, the person preparing the registry entry is taken to the underlying document for further review. In some example approaches, clicking on the confidence level in the context entry in context display box 58 causes system 10 to display the factors that went into the confidence level calculation.

In one example approach, when the person preparing the registry entry selects “Save and Close” or “Reviewed,” a check is made to determine if there are any conflicting entries. If so, an error message is displayed. The person preparing the registry entry may simply address the errors and indicate either “Save and Close” or “Reviewed” when finished.

In one example approach, a properly defined natural language processor 24 and a properly defined rule-based processor 26 are used to analyze and annotate a corpus of medical documents based on the information relevant to a medical registry. For instance, natural language processor 24 may identify all variations in the documents of “long QT syndrome,” and the context in which the variation was found. Rule-based processor 26 may identify all information in the documents that can be used to determine “long QT syndrome,” and the context in which the information was found. For instance, as noted above, there may be a rule equating a QT interval greater than 460 msec as an indicator of long QT syndrome and a rule determining that one or more diagnostic codes or one or more billing codes are indicators of long QT syndrome. The QT interval value is stored with the context in which it was found and each diagnostic or billing code indicating long QT syndrome is stored with the context in which it was found. Each document is annotated to reflect the registry relevant information found in the document and the annotated documents are used to train machine learning processor 22, as will be detailed below.

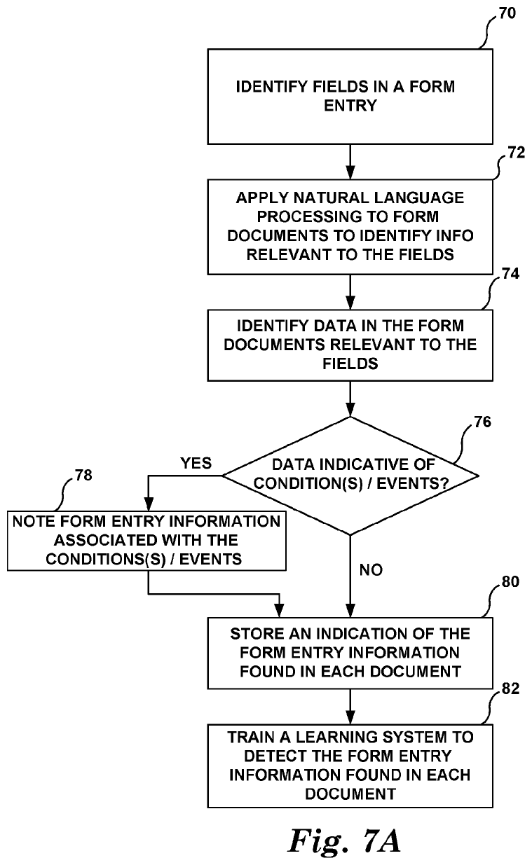

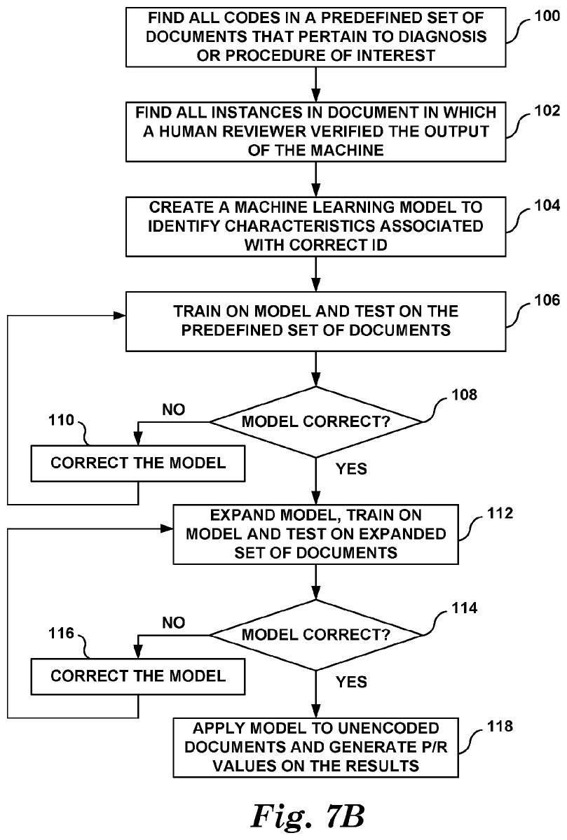

FIG. 7A illustrates an example method of training a machine learning processing system to analyze and extract information from documents. In the example shown in FIG. 7A, a document analysis and data extraction system 10 identifies information needed to submit a form entry, for example, to submit a registry entry to a registry. (70) In one example approach, an analyst identifies the information needed to submit a form entry 16 and enters the information needed into system 10. A natural language processor 24 in system 10 then processes a representative sample of documents to identify instances of the identified form entry information in the documents in the sample. (72) The natural language processor 24 also identifies instances of information (such as key words or test results) in the documents that can be used to derive analyst identified form entry information. (74) A rule-based processor 26 in system 10 receives the information from natural language processor 24 and determines if form relevant information can be derived from the data. (76) If so, rule-based processor 26 highlights the data in the document as form entry relevant information. (78) System 10 annotates and stores versions of each document with indications of the medical entry information identified or derived from each document. (80) System 10 trains machine learning processor 22 to detect the form entry information based on the form entry information identified or derived from each document. (82)

The quality of the documents used to train machine learning processor 22 is important. In one example approach, analysts process documents in the representative sample of documents to remove errors and omissions before using the documents to train the machine learning system. If the document database is too extensive for analyst review, random samples from database 14 can be reviewed for accuracy as a quality check of the training. In another example approach, a human-curated set of documents is used in the initial training of machine learning processor 22. The entire corpus of documents is then reviewed via the methods of FIG. 2 to identify documents that contain relevant errors.

FIG. 7B illustrates an example method of training a machine learning processing system to analyze and extract information from documents, in one example, medical documentation. In the example shown in FIG. 7B, an analyst identifies all ICD-10-CM/PCS codes that pertain to a diagnosis or procedure of interest. (100) Then the analyst identifies documents in a predefined set of documents in which instances of a diagnosis or a procedure of interest identified at 100 were found in the document and in which a human reviewer verified the output (agreed/accepted as truth) made by the machine that analyzed the document. (102) The analyst then creates a machine learning model to identify characteristics associated with the ‘correct identification of the disease or syndrome (compare documents of those with confirmed condition and diagnosis vs those with absence of condition and diagnosis). (104) The machine learning model is used to train machine learning processor 22 and the machine learning processor 22 analyzes the predefined set of documents (or other known set of documents) based on the training. A check is made at 108 to determine if the model used identified an acceptable number of instances of the diagnoses and procedures of interest identified at 100. If not, the model is corrected (110) and used at 106 to train machine learning processor 22. If the machine learning model used at 106 identified an acceptable number of instances of the diagnoses and procedures of interest identified at 100, expand the model, train processor 22 on the model and test on an expanded set of annotated documents. (112)

A check is made at 114 to determine if the expanded machine learning model used identified, in the expanded set of documents, an acceptable number of instances of the diagnoses and procedures of interest identified at 100. If not, the model is corrected (116) and used at 112 to train machine learning processor 22. If the machine learning model used at 112 identified, in the expanded set of documents, an acceptable number of instances of the diagnoses and procedures of interest identified at 100, apply the expanded model to documents that are not annotated. (118) In one example approach, the expanded model includes programming code for generating a confidence level for each determination of an instance of the diagnoses and procedures of interest identified at 100. A person preparing a medical entry may use the confidence levels to help them select between conflicting data.

In one example approach, a properly defined natural language processor 24 and a properly defined rule-based processor 26 are used to analyze and annotate a corpus of medical documents based on diagnostic codes. The diagnostic codes are then mapped to fields in a medical entry and exported to be used as input for the medical entry. For instance, natural language processor 24 may identify all variations in the documents of “long QT syndrome,” and the context in which the variation was found. Rule-based processor 26 may identify all information in the documents that can be used to determine “long QT syndrome,” and the context in which the information was found. Each document is annotated with the diagnostic code for long QT syndrome and, in some example approaches, the context in which information leading to the diagnostic code was found. The diagnostic codes in the annotated documents are then used to develop the medical entries and, in some example approaches, to train machine learning processor 22. Such an approach simplifies the mapping of conditions to medical entry fields in system 10.

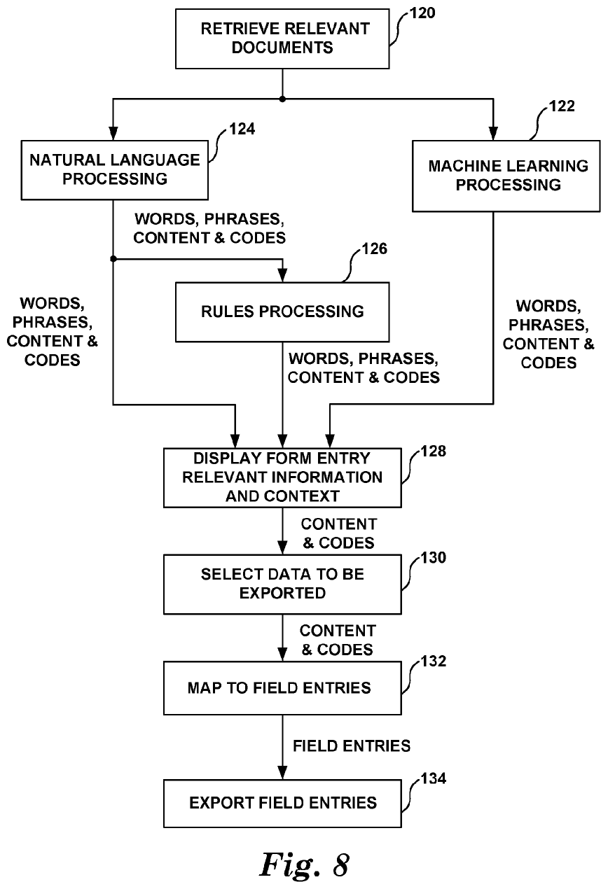

FIG. 8 illustrates an example method of analyzing patient medical documentation based on natural language processing, rule-based processing and machine learning to extract information relevant to medical entry data collection forms. In the example shown in FIG. 8, system 10 retrieves medical documents associated with a patient. (100) Machine learning processor 22 analyzes the documents and identifies medical entry relevant information based on the machine learning model with which it was trained. (122) In one example approach, machine learning processor 22 outputs words, phrases, content and codes used to identify medical entry relevant information found during its analysis and to annotate the document to reflect the presence of the medical entry relevant information. In some example approaches, the content includes sections of document used to provide the context in which the medical entry relevant information was found.

In the example shown in FIG. 8, natural language processor 24 analyzes the documents and identifies medical entry relevant information based on an analysis of key words and phrases. (122) In one example approach, natural language processor 24 outputs words, phrases, content and codes used to identify form entry relevant information found during its analysis and to annotate the document to reflect the presence of the form entry relevant information. In some example approaches, the content includes sections of document used to provide the context in which the form entry relevant information was found.

In one example, natural language processor 24 also outputs words, phrases, content and codes used by rule-based processor 26 to derive medical entry relevant information. In some example approaches, the content includes sections of document used to provide the context in which the medical entry relevant information was found.

In the example shown in FIG. 8, rule-based processor 26 receives, from natural language processor 24, indications of data found in each document that may be used to derive data relevant to one or more form entries 16. In some example approaches, the data includes sections of document used to provide the context in which the data was found. Rule-based processor 26 derives data relevant to one or more form entries 16 from the information received from natural language processor 24. In some example approaches, the content includes sections of document used to provide the context in which the derived medical entry relevant information was found.

The form entry relevant information is displayed. (128) In some example approaches, only selected form entry relevant information is displayed. For example, display 28 may, in the presence of multiple versions of the same information, display only those versions with the highest confidence ratings. Optionally, the person preparing the form entry selects the data to be exported. (130) The data to be exported is mapped to the form field entries (132) and exported as form field entries (134). In some example approaches, the exported form field entries are transferred directly to a form entry editor for inclusion in the form entry.

In one example approach, natural language processor 24 analyzes and annotates each document or component of data, such as laboratory, case documents, or test results. In the example of medical entries, this includes identifying and tagging within every document or data source each diagnosis, symptom, vital sign, or other patient information, as well as each test, lab, or procedure performed. In some example approaches, natural language processor 24 also determines whether each element identified is current for the visit or encounter, or whether it is historical (from a past encounter), or is related to a familial history or linkage. In the medical entry example, each relevant piece of information about a patient's current, historic, or familial medical history is then mapped by natural language processor 22 to a concept identification code. The concept identification code is an intermediary code set that is mapped to and from other commonly used code sets. These common identifier codes for each patient, along with the relationships between each common identifier codes, are then stored in the case model as well.

In one example approach, the common identification codes are part of a healthcare data dictionary (HDD). Each of the concept identification codes is mapped, or linked, to most other available industry coding sets or terminology standards, such as ICD-9 and 10 codes or SNOMED-CT codes. Mapping every piece of information in a patient's medical record to a concept identification code, allows for ready translation of any one code or term, to any other code or term from another standard.

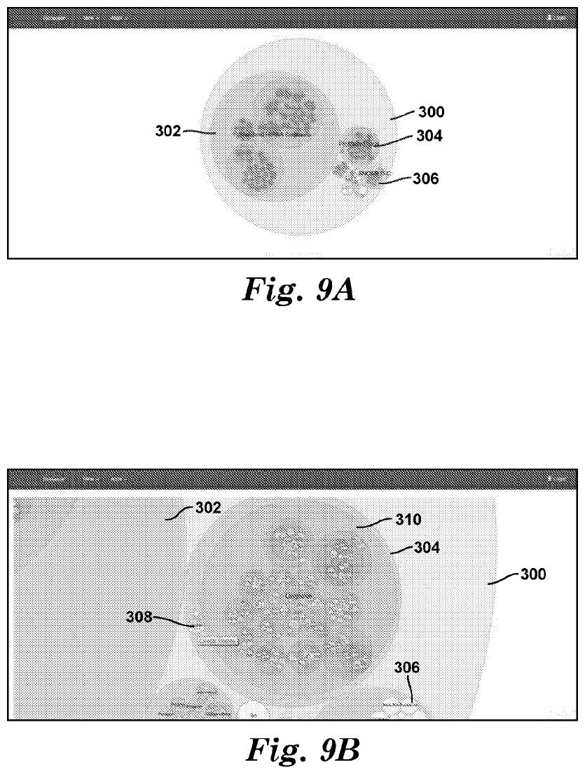

FIGS. 9A and 9B illustrate an organization of clinical and diagnostic codes in one example of a Healthcare Data Dictionary (HDD). In the example shown in FIGS. 9A and 9B, the universe 300 of HDD codes includes standard HIPAA codesets 302, problem diagnostic codes 304, SNOMED CT codes 306 and assorted other codesets. As is shown in FIG. 9B, problem diagnostics codes 304 include clinical findings codes 308 and diagnosis codes 310. As noted above, in one example approach, the medical entry relevant information is associated with one or more diagnostic or clinical codes selected from the HDD and the medical entry relevant information identified and selected is mapped as a function of the diagnostic codes to medical field entries before being exported. As noted above, such an approach simplifies adding or changing codes associated with clinical conditions.

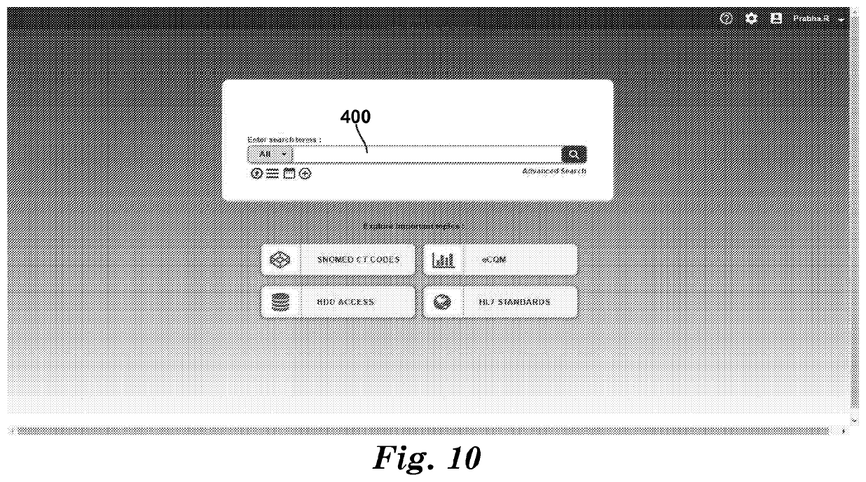

FIG. 10 illustrates an example user interface used select documents used to train machine learning processor 22. In one example approach, search box 400 is used to select documents to be used to train machine learning processor 22.

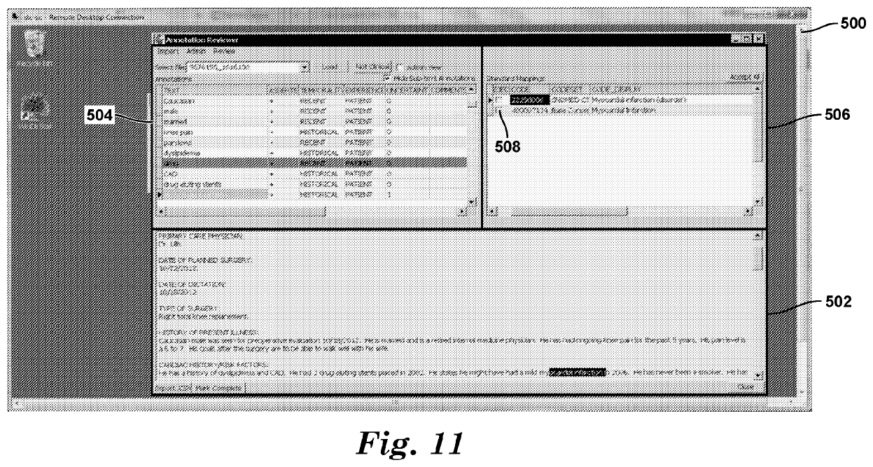

FIGS. 11-13 illustrate example user interfaces used to analyze and annotate documents used to train the machine learning processor. In the example approach of FIG. 11, user interface 500 includes a document viewer window 502, a text context window 504 and a standard mappings window 506. In the example shown in FIG. 11, the phrase “myocardial infarction” has been found in a document and system 10 displays context such as the temporality of the information and the individual involved in text context window 504. System 10 also displays representative code mappings in standard mappings box 506. The person annotating this document can select either the coding from the SNOMED CT coding system or the coding from the base concept coding system and does so by checking the box 508 before the coding selection.

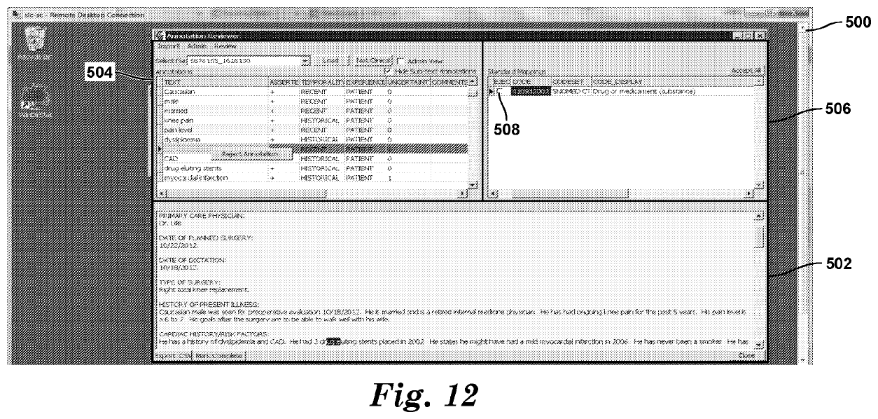

In the example approach shown in FIG. 12, the word “drug” has been found in a document and system 10 displays context such as the temporality of the information and the individual involved in text context window 504. System 10 also displays representative code mappings in standard mappings box 506. In this example, however, the word “drug” in the document being examined is not an indicator of the “drug or medicament (substance)” associated with SNOMED code 410942007. The person annotating this document therefore rejects the coding and the subsequent annotation of the word “drug”.

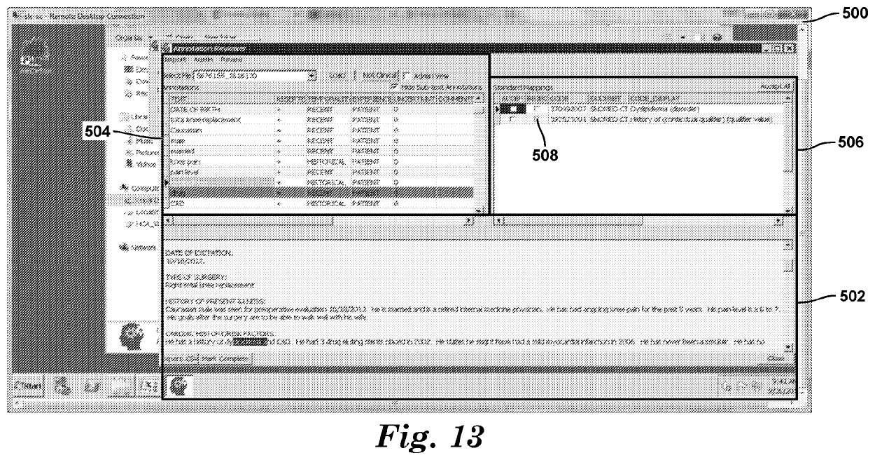

In the example approach shown in FIG. 13, the word “dyslipidemia” has been found in a document and system 10 displays context such as the temporality of the information and the individual involved in text context window 504. System 10 also displays representative code mappings in standard mappings box 506. In this example, however, the word “dyslipidemia” in the document being examined may be mapped to two different SNOMED codes. The person annotating this document selects an appropriate mapping by clicking one of the check boxes 508 and the document is annotated accordingly.

FIG. 14A illustrates an example flow diagram to extract evidence and context for a form field entry, implemented by a computer system. Some of the steps are optional. First, the computer system parses a document into sections. (1410) For each sentence in a section (1415), the computer system takes a sequence of steps in 1420-1450 described below to determine relevant context/evidence, where some steps are optional and the steps are not required in the order described. First, search a form field entry relevant phrase (1420). If a form field entry relevant phrase is found, the system will search evidence/context trigger terms, which includes negation trigger terms (1430), temporality trigger terms (1440), and/or other trigger terms. For each trigger term found, the effect and scope of the trigger term will be determined. For example, for a negation trigger term (1432), the system determines whether it is a pre-negation trigger term (1434) (e.g., no signs and symptoms of heart failure), a post-negation trigger term (1435) (e.g., heart failure was not evident upon exam), a conjunction term (1436) (e.g., excluded pneumonia and heart failure after examination), or another type of negation trigger term. Based on the type of the trigger term, the system will determine the scope of negation (1430). As another example, the system will determine the scope of a temporality trigger term (1444) (e.g., “patient has a history of atrial fibrillation, diabetes, cardiomyopathy, chronic pain and congestive heart failure” vs. “diagnosed with congestive heart failure recently”). After the scope and effect of one or more trigger terms are determined, the system will determine the context/evidence for the form field entry (1450), as illustrated in FIG. 14B.

In the examples illustrated in FIGS. 14B and 14C, the computer system displays the form field entry, the number of supporting evidences and the number of negating evidences on the graphical user interface. In some cases, the computer system displays the contexts and evidences of the form field entry. The context/evidences include supporting evidence(s) and/or negating evidence(s). In some cases, the context/evidences include one or more temporality evidences.

FIG. 14D shows an example user interface allowing a user to select or determine the form field entry, if different from what is determined by the computer system, and to provide inputs and/or comments for the form field entry. FIG. 14E shows an example user interface allowing a user to open the entire document and review the contexts/evidences, and optionally, copying selected contexts/evidences if different from the system selection.

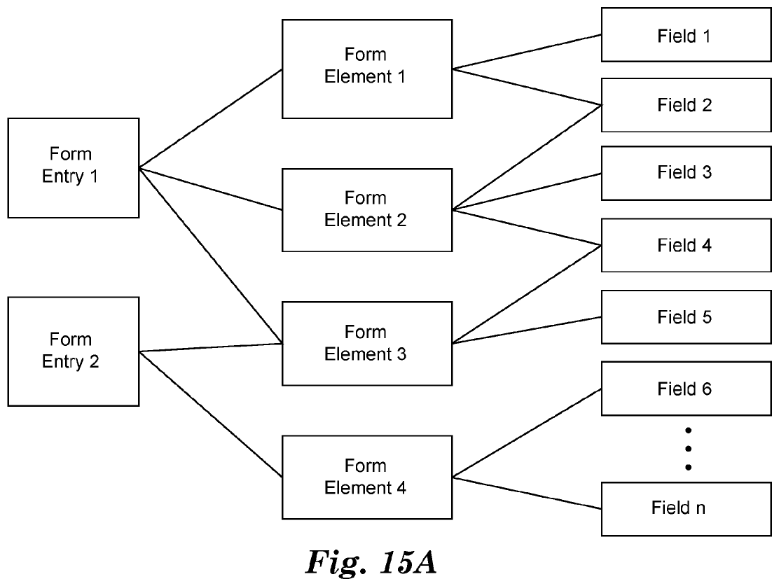

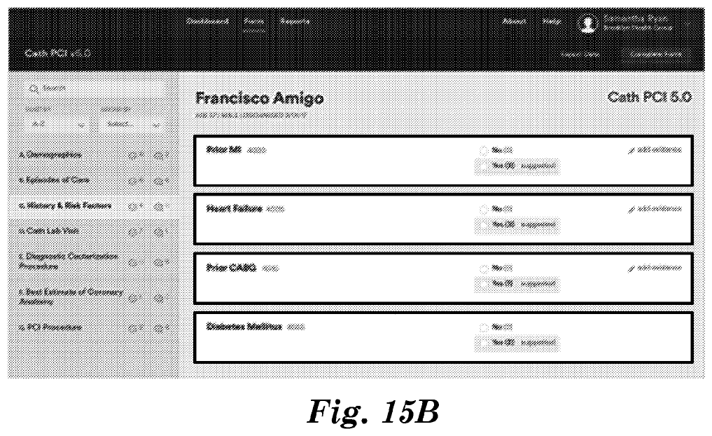

FIG. 15A illustrates an example data structure that can be used to represent form entries and form entry fields. In this example, a form entry includes one or more form elements and a form element includes one or more fields, which is also referred to as form field entry in this disclosure. In some cases, two form entries may include a same form element. In some cases, two form elements may include a same field. In some cases, each field is stored in a data structure including the value of the field and the context/evidence(s) of the field.

FIG. 15B illustrates an example user interface for managing forms. The left column shows a list of form elements, and a number of supporting evidences, a number of negating evidences for the respective form element. The right window shows the form field entries for the selected form element.

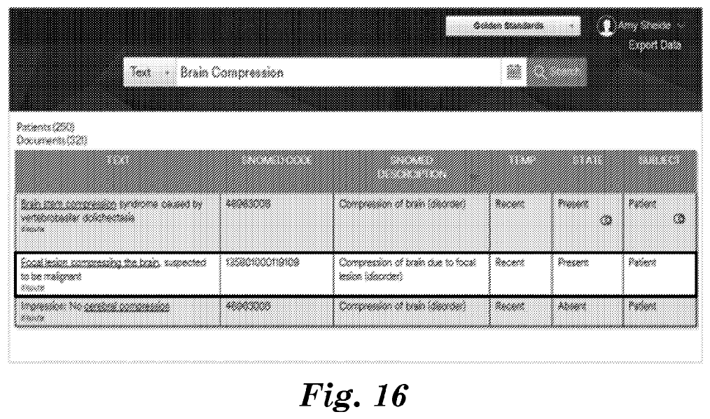

In some embodiments, the computer system may utilize a dictionary to facilitate search capability. In one example, the dictionary is a healthcare data dictionary. FIG. 16 illustrates an example user interface allowing a user to enter a search term to do a text search. In some cases, the computer system may use a dictionary to identify relevant search phrases, which includes the search term alternative terms and/or codes for the search term. In some cases, the computer system may search the records using the identified search phrases. In some cases, the system identifies one or more documents containing the search phrases and displays a link to the documents on the user interface.

Embodiments of system 10 described herein can review all documentation available for a patient's case and identify all relevant problems, diagnoses, and issues that a patient is being treated for, or that are associated with this patient's medical condition (herein referred to collectively as “problems”). These problems may, in some embodiments, be coded per standards consistent with the International Classification of Disease or other industry standards (for example, ICD-9, ICD-10, or SNOMED CT), and consistent with the notion of Meaningful Use as defined by the Health Information Technology for Economic and Clinical Health (HITECH) Act of 2009 and administered by the Centers for Medicare and Medicaid Services (CMS). Meaningful Use is related to the Medicare EHR Incentive Program which provides incentive payments to eligible professionals, eligible hospitals, and CAHs that demonstrate meaningful use of certified EHR technology. Consistent with embodiments further described herein, aspects of these automatically identified problems, diagnoses, and issues are used to populate medical entry fields for submission.

### EXEMPLARY EMBODIMENTS

### Embodiment A1

A method of extracting medical entry information from medical documentation, the method comprising:

identifying patient information needed for a predefined medical entry;

finding the patient information in documents associated with the patient, wherein finding the patient information includes annotating the documents with a natural language processor to detect phrases and words corresponding to the patient information in the patient documents and analyzing the documents with a machine learning processor trained using the annotated documents to detect the patient information in the patient documents; and exporting the patient information found as medical entry fields.

### Embodiment A2

The method of Embodiment A1, wherein finding the patient information further includes analyzing the documents with a rule-based processor to derive patient information from information stored in the patient documents.

### Embodiment A3

This method of Embodiment A2, wherein the rule-based processor is configured to derive patient information from the annotated documents by applying rules of medical information interpretation.

### Embodiment A4

A method of extracting medical entry information from medical documentation, the method comprising: identifying patient information needed for a predefined medical entry; finding the patient information in documents associated with the patient, wherein finding the patient information includes analyzing the documents with a machine learning processor trained using annotated documents to detect the patient information in the patient documents; displaying the patient information; receiving input selecting the patient information to export; and exporting the selected patient information as medical entry fields.

### Embodiment A5

The method of Embodiment A4, wherein finding the patient information further includes analyzing the documents with a natural language processor to detect phrases and words corresponding to the patient information in the patient documents, wherein finding the patient information includes analyzing the documents with a machine learning processor trained detect the patient information in patient documents.

### Embodiment A6

The method of Embodiment A5, wherein finding the patient information further includes analyzing the documents with a rule-based processor to derive patient information from information stored in the patient documents.

### Embodiment A7

A method of training a machine learning processor, comprising: identifying, in a predefined set of documents annotated with codes, all the codes that pertain to diagnoses or procedures of interest; identifying all instances in the predefined set of documents of the identified codes; creating a machine learning model to identify characteristics of each document that led to annotation of the document with the identified codes; training the machine learning processor based on the machine learning model; and testing the machine learning processor on the predefined set of documents.

### Embodiment A8

The method of Embodiment A7, wherein the method further comprises applying the machine learning processor to an expanded set of annotated documents and verifying the results.

### Embodiment A9

The method of Embodiment A7 or A8, wherein the method further comprises applying the machine learning model to unannotated documents via the machine learning processor and verifying the results.

### Embodiment A10

The method of any one of Embodiments A7-A9, wherein the method further comprises applying the machine learning processor to an expanded set of annotated documents and retraining the machine learning processor as a function of the results.

### Embodiment A11

The method of Embodiment A10, wherein the method further comprises applying the machine learning model to unannotated documents via the machine learning processor and verifying the results.

### Embodiment A12

A method of training a machine learning processor, comprising:

identifying fields in a medical entry; applying natural language processing to annotate information relevant to the medical entry fields in a pre-defined set of documents; applying rule-based processing to annotate information used to derive information relevant to the medical entry fields in a pre-defined set of documents; and training the machine learning processor as a function of the annotated information.

### Embodiment A13

The method of Embodiment A12, wherein the method further comprises applying the machine learning processor to the pre-defined set of documents and verifying the results.

### Embodiment A14

A method of training a machine learning processor, comprising: identifying fields in a medical entry; applying natural language processing to annotate information relevant to the medical entry fields in a pre-defined set of documents; applying rule-based processing to annotate information used to derive information relevant to the medical entry fields in a pre-defined set of documents; pretraining the machine learning processor as a function of the annotated information; and verifying the pretrained machine learning processor against the pre-defined set of documents.

### Embodiment A15

The method of Embodiment A14, wherein the method further comprises applying the machine learning processor to a second pre-defined set of annotated documents and verifying the results.

### Embodiment A16

The method of Embodiment A14 or A15, wherein the method further comprises: applying the machine learning processor to a second pre-defined set of annotated documents; verifying the results; and retraining the machine learning processor based on the results.

### Embodiment A17

A computer system having at least one processor and memory comprising functional modules programmed to carry out the methods in any of Embodiments A1-A16.

### Embodiment A18

A non-transient computer readable medium having instructions that, when executed by a computer system, cause the computer system to carry out the methods described in any of Embodiments A1-A16.

### Embodiment B1

A method of extracting form entries, the method comprising: receiving one or more documents; identifying fields, by a processor, needed for a predefined form entry; and generating a plurality of field records based on the documents, wherein each of the plurality of field records corresponds to one of the fields and includes a field value and one or more evidences, wherein for each of the plurality of field records, extracting the one or more evidences from the documents with a natural language processor to detect phrases and words corresponding to the field in the documents; analyzing the one or more evidences with a machine learning processor; and suggesting the field value based on the one or more evidences, wherein at least one of the one or more evidences is a negating evidence.

### Embodiment B2

The method of Embodiment B1, further comprising: exporting the plurality of field records.

### Embodiment B3

The method of Embodiment B1 or B2, wherein at least one of the one or more evidences in one of the plurality of field records is a temporality evidence.

### Embodiment B4

The method of any one of Embodiments B1-B3, wherein at least one of the one or more evidences in one of the plurality of field records is a subject evidence that is related to the subject of the documents.

### Embodiment B5

The method of any one of Embodiments B1-B4, wherein at least one of the one or more evidences in one of the plurality of field records is a supporting evidence.

### Embodiment B6

The method of Embodiment B5, further comprising: displaying the plurality of field records.

### Embodiment B7

The method of Embodiment B5, wherein displaying the plurality of field records comprises displaying the determined field content, a number of supporting evidences, and a number of negating evidences.

### Embodiment B8

The method of Embodiment B5, wherein displaying the plurality of field records comprises providing a document link for at least one of the one or more evidences in one of the plurality of field records.

### Embodiment B9

The method of any one of Embodiments B1-B8, further comprising: identifying form elements, wherein each of the form elements comprises one or more fields; and generating a plurality of form element records, wherein each of the plurality of form element records comprises one or more field records corresponding to the one or more constituent fields.

### Embodiment B10

The method of any one of Embodiment B1-B9, further comprising: receiving a search term from a user interface; selecting a plurality of search phases based on the search term using a dictionary; identifying a plurality of documents containing relevant search results using the plurality of search phases and the plurality of field records.

### Embodiment B11

A computer system having at least one processor and memory comprising functional modules programmed to carry out the methods in any of Embodiments B1-B10.

### Embodiment B12

A non-transient computer readable medium having instructions that, when executed by a computer system, cause the computer system to carry out the methods described in any of Embodiments B1-B11.

The methods thus described may be implemented on one or more computing systems having processors and memories. Non-transient computer readable media may also include instructions that cause such systems to carry out methods described above.

Various modifications and alterations of this invention will be apparent to those skilled in the art without departing from the spirit and scope of this invention. The inventions described herein are not limited to the illustrative examples set forth herein. For example, the reader should assume that features of one disclosed example can also be applied to all other disclosed examples unless otherwise indicated. It should also be understood that all U.S. patents, patent application publications, and other patent and non-patent documents referred to herein are incorporated by reference, to the extent they do not contradict the foregoing disclosure.

What is claimed is:

1. A method of extracting form entries, the method comprising:
receiving one or more documents;
identifying fields, by a processor, needed for a predefined form entry; and
generating a plurality of field records based on the documents, wherein each of the plurality of field records corresponds to one of the fields and includes a field value and one or more evidences, wherein for each of the plurality of field records,
extracting the one or more evidences from the documents with a natural language processor to detect phrases and words corresponding to the field in the documents;
analyzing the one or more evidences with a machine learning processor; and
suggesting the field value based on the one or more evidences,
wherein at least one of the one or more evidences is a negating evidence.

2. The method of claim 1, wherein at least one of the one or more evidences in one of the plurality of field records is a temporality evidence.

3. The method of claim 1, wherein at least one of the one or more evidences in one of the plurality of field records is a subject evidence that is related to the subject of the documents.

4. The method of claim 1, wherein at least one of the one or more evidences in one of the plurality of field records is a supporting evidence.

5. The method of claim 4, further comprising:
displaying the plurality of field records.

6. The method of claim 4, wherein displaying the plurality of field records comprises displaying the determined field content, a number of supporting evidences, and a number of negating evidences.

7. The method of claim 4, wherein displaying the plurality of field records comprises providing a document link for at least one of the one or more evidences in one of the plurality of field records.

8. The method of claim 4, further comprising:
receiving a user input regarding one of the fields for the predefined form entry; and
updating the corresponding field record with the input.

9. The method of claim 1, further comprising:
identifying form elements, wherein each of the form elements comprises one or more fields; and
generating a plurality of form element records, wherein each of the plurality of form element records comprises one or more field records corresponding to the one or more constituent fields.

10. The method of claim 1, further comprising:
receiving a search term from a user interface;
selecting a plurality of search phases based on the search term using a dictionary;
identifying a plurality of documents containing relevant search results using the plurality of search phases and the plurality of field records.

11. A method of extracting medical entry information from medical documentation, the method comprising:
identifying patient information needed for a predefined medical entry;
finding the patient information in documents associated with the patient, wherein finding the patient information includes annotating the documents with a natural language processor to detect phrases and words corresponding to the patient information in the patient documents and analyzing the documents with a machine learning processor trained using the annotated documents to detect the patient information in the patient documents; and
exporting the patient information found as medical entry fields.

12. The method of claim 1, wherein finding the patient information further includes analyzing the documents with a rule-based processor to derive patient information from information stored in the patient documents.

13. This method of claim 12, wherein the rule-based processor is configured to derive patient information from the annotated documents by applying rules of medical information interpretation.

14. A method of extracting medical entry information from medical documentation, the method comprising:
identifying patient information needed for a predefined medical entry;
finding the patient information in documents associated with the patient, wherein finding the patient information includes analyzing the documents with a machine learning processor trained using annotated documents to detect the patient information in the patient documents;
displaying the patient information;
receiving input selecting the patient information to export; and
exporting the selected patient information as medical entry fields.

15. The method of claim 14, wherein finding the patient information further includes analyzing the documents with a natural language processor to detect phrases and words corresponding to the patient information in the patient documents, wherein finding the patient information includes analyzing the documents with a machine learning processor trained detect the patient information in patient documents.
